(19) United States
(12) Patent Application Publication
(10) Pub. No.: US 2008/0317819 A1
(43) Pub. Date: Dec. 25, 2008

(54) Iop lowering drug combination or non-combination loaded contact lens with zonal drug delivery areas

(76) Inventors: Werhner C. Orilla, Anaheim, CA (US); Michael Robinson, Irvine, CA (US); James Burke, Santa Ana, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/821,085

(22) Filed: Jun. 21, 2007

(51) Int. Cl. G02C 7/04; A61K 31/502; A61P 27/06

(52) U.S. Cl. 424/429; 514/249

(57) ABSTRACT

A contact lens for delivery of a multiplicity of drugs includes a lens body with a first drug incorporated in a first zonal area of the lens body and a second drug incorporated in a second zonal area of the lens body. The first and second drugs are dissimilar and the first and second zonal areas are spaced apart from one another.

The present invention is generally related to a delivery vehicle for dispensing therapeutic drugs and is more particularly directed to a biocompatible contact lens for delivery of a multiplicity of drugs.

Drug loaded contact lenses have been described in the art, see for example, U.S. Ser. No. 11/594,518 to Orilla, Burke, and Robinson. This application is to be incorporated herein in its entirety by this specific reference thereto.

Glaucoma is an ocular disorder associated with elevated intraocular pressure (IOP) which is too high for normal ocular physiology and may result in irreversible loss of visual function. Owing to the progressive nature of glaucoma, the disease may begin with elevated IOP, progress through loss of visual field and eventually lead to blindness. Ocular hypertension, i.e., the condition of elevated intraocular pressure without optic nerve head damage or characteristic glaucomatous visual field defects, is now believed by many ophthalmologists to represent the earliest stage of glaucoma.

It has been found that administration of one or more prostaglandins in combination with one ore more brimonidine derivatives controls or lowers intraocular pressure (IOP) without accompanying inflammatory response (including hyperemia) typically found with topical use of prostaglandins, see U.S. Pat. No. 6,294,563. This patent is to be incorporated herein in its entirety for the prior of teaching the use of drug to lower IOP. Additionally, it has been found that brimonidine derivatives exert a neuroprotective effect on the eye reducing the potential degeneration of nerves; for example, the optic nerve.

### SUMMARY OF THE INVENTION

The present invention provides a contact lens and method useful for the treatment of glaucoma and ocular hypertension which also provide neuroprotection to the optic nerve. Thus, the present invention has a combined effect of alleviating symptoms of glaucoma (reducing, intraocular pressure) as well as helping prevent progression of optic nerve degeneration.

More specifically, a contact lens in accordance with the present invention for the delivery of a multiplicity of drugs generally includes a lens body having an optical axis.

A first drug is incorporated in a first zonal area of the lens body and a second drug is incorporated into a second zonal area of the lens body. The first and second drugs are dissimilar and further the first and second zonal areas are spaced apart from one another.

In one embodiment of the present invention, the first and second zonal areas are concentric rings and in another embodiment of the present invention, each of the concentric rings are segmented.

More particularly, as hereinabove noted, the first and second drugs may comprise different intraocular pressure lowering drugs and the first and second zonal areas may be disposed in locations within the lens body to enable enhanced drug efficiency and lowered side effects.

The first zonal area may comprise an inner ring and the second zonal area may comprise an outer ring and the first drug is disposed in the inner ring may comprise prostaglandin and the second drug disposed in the outer ring may comprise a brimonidine derivative.

In addition, the contact lens may also include a centered clear area disposed about an optical axis for enhancing optical acuity.

The concentric rings may also function to provide a simulated iris, or iris pattern, on an eye.

A method for treating a mammal suffering from glaucoma, or ocular hypertension, in accordance with the present invention includes disposing a contact lens onto an eye of the mammal with the contact lens including a lens body having an optical axis, a prostaglandin incorporated into a first zonal area of the lens body, a brimonidine derivative incorporated in a second zonal area of the lens body with the first and second zonal areas being spaced apart from one another.

### BRIEF DESCRIPTION OF THE DRAWINGS

The advantages and features of the present invention will be better understood by the following description when considered in conjunction with the accompanying drawings, in which:

FIG. 1 is a plan view representing a contact lens for delivery of a multiplicity of drugs through a multiplicity of zonal areas on a lens body with the zonal areas being spaced apart from one another and in the embodiment shown in the form of concentric rings; and

FIG. 2 is another embodiment of the present invention showing the contact lens utilizing a multiplicity of zonal areas for delivery of dissimilar drugs with the zones being in the form of segmented rings spaced apart from one another.

### DETAILED DESCRIPTION

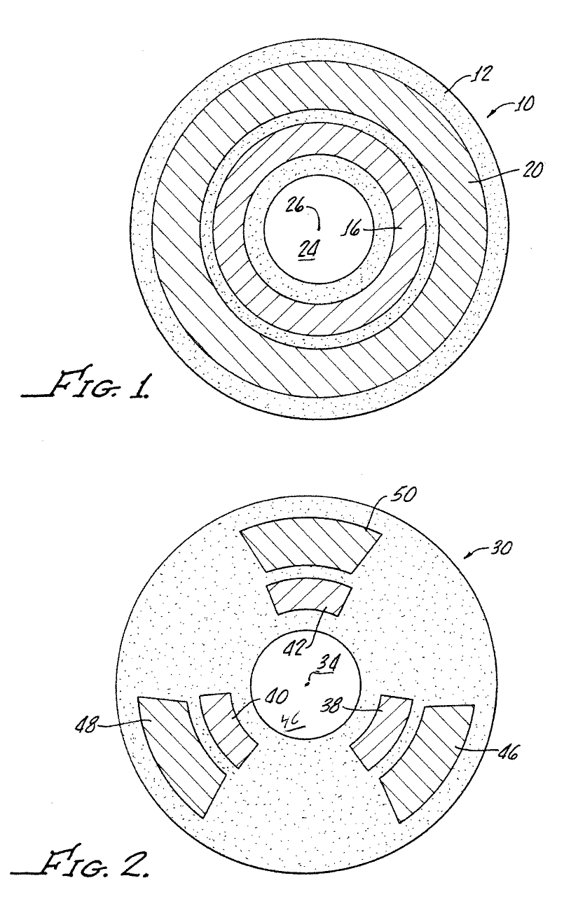

With reference to FIG. 1 there is shown a contact lens 10 including a lens body 12 which may be formed from any suitable well-known material. A first zonal area, which may be an inner ring, 16, incorporating a first drug, preferably a prostaglandin, or prostamide, is disposed in a spaced apart relationship with a second zonal area, which may be an outer ring, 20 incorporating preferably a brimonidine derivative. As hereinabove noted, these drugs are suitable in combination for treating a mammal suffering from glaucoma or ocular hypertension and the method of the present invention for treating mammals (not shown) suffering from glaucoma or ocular hypertension includes disposing the contact lens 10 onto an eye, not shown, of the mammal.

A clear area 24 may be disposed about an optical axis 26 for enabling unrestricted vision through the contact lens 10 and the inner and outer rings 16, 20 also provide an iris-like pattern when disposed on the mammal's eye. The prostaglandin and brimonidine compounds are described in U.S. Pat. No. 6,294,563 which has been incorporated into the present application and accordingly no further elucidation of these drugs is presented here for brevity.

It should further be appreciated that while prostaglandin and brimonidine are specifically set forth, other drug combinations of dissimilar drugs may be utilized in accordance with the present invention.

It should also be appreciated that the inner ring 16 location of the prostaglandin will allow less drug to be diffused into the conjunctiva area (not shown) of an eye (not shown) thus allowing a lesser chance of hyperemia.

With reference to FIG. 2, there is shown an alternative embodiment to the contact lens 30 having a lens body 32 with an optical axis 34.

In this embodiment 30, spaced apart inner zonal areas 38, 40, 42 comprising an inner segmented ring have a prostaglandin, or prostamide, incorporated therein. Outer zonal areas 46, 48, 50 incorporate brimonidine and, as shown, form an outer segmented ring which is spaced apart from the inner segment, or zones, 38, 40, 42.

Operation and usefulness of the embodiment 30 is similar to the embodiment 10 hereinabove described. Materials of construction as hereinabove noted are well known in the art.

A clear area 46 may be disposed upon the optical axis 34, as hereinabove described in connection with the embodiment 10 shown in FIG. 1.

While shown in a symmetric pattern in FIG. 2, the zones 38, 40, 42, 46, 48, 50 may be disposed superiorly since this may preferably lower IOP by improved distribution of drug through the corner (not shown) and into the anterior chamber (not shown). In this embodiment, the lens body 12 may be weighted, have varying thickness, or a prism ballast system (not shown) in order to provide rotational stability.

Although there has been hereinabove described a specific IOP lowering drug combination or non-combination loaded contact lens with zonal drug delivery areas in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A contact lens for delivery of a multiplicity of drugs, said contact lens:
a lens body having an optical axis;
a first drug incorporated in a first zonal area of said lens body;
a second drug incorporated in a second zonal area of said lens body, the first and second drug being dissimilar; and
the first and second zonal areas being spaced apart from one another.

2. The contact lens according to claim 1 wherein the first and second zonal areas are concentric rings.

3. The contact lens according to claim 2 wherein each of the first and second zone areas are segmented.

4. The contact lens according to claim 1 wherein the first and second drugs comprises different intraocular pressure lowering drugs.

5. The contact lens according to claim 1 wherein the first and second zonal areas are disposed at locations within said lens body to enable enhanced drug efficiency and lowered side effects.

6. The contact lens according to claim 2 wherein the first zonal area comprises an inner ring and the second zonal area comprises an outer ring.

7. The contact lens according to claim 6 wherein said first drug comprises prostaglandin and said second drug comprises a brimonidine derivative.

8. The contact lens according to claim 3 wherein the first zonal area comprises an inner segmented ring and the second zonal area comprises an outer segmented ring.

9. The contact lens according to claim 8 wherein said first drug comprises a prostaglandin and the second drug comprises a brimonidine derivative.

10. A contact lens for delivery of a multiplicity of drugs, said contact lens:
a lens body having an optical axis;
a centered clear area disposed about said optical axis;
a first drug incorporated in a first zonal area of said lens body;
a second drug incorporated in a second zonal area of said lens body, the first and second drug being dissimilar; and
the first and second zonal areas being spaced apart from one another.

11. The contact lens according to claim 10 wherein the first and second zonal areas are concentric rings.

12. The contact lens according to claim 14 wherein each of the first and second zone areas are segmented.

13. The contact lens according to claim 10 wherein comprises different intraocular pressure lowering drugs.

14. The contact lens according to claim 1 wherein the first and second zonal areas are disposed at locations within said contact lens being to enable enhanced drug efficiency and lowered side effects.

15. The contact lens according to claim 11 wherein the first zonal area comprises an inner ring and the second zonal area comprises an outer ring.

16. The contact lens according to claim 15 wherein said first drug comprises prostaglandin and said second drug comprises a brimonidine derivative.

17. The contact lens according to claim 11 wherein the first zonal area comprises an inner segmented ring and the second zonal area comprises an outer segmented ring.

18. The contact lens according to claim 16 wherein said first drug comprises a prostaglandin and said second drug comprises a brimonidine derivative.

19. A method for heating a mammal suffering from glaucoma or ocular hypertension, said method comprising disposing a contact lens unto an eye of the mammal, said contact lens comprising:
a lens body having an optical axis;
a prostaglandin incorporated in a first zonal area of said lens body;
a brimonidine derivative incorporated in a second zonal area of said lens body; and
the first and second zonal areas being spaced apart from one another.
